(19) United States
(12) Patent Application Publication
(10) Pub. No.: US 2012/0065527 A1
(43) Pub. Date: Mar. 15, 2012

(54) Methods and Systems for Monitoring Aterial Stiffness

(75) Inventors: Jong Gill, Valencia, CA (US); Brian Jeffrey Wenzel, San Jose, CA (US); Allen Keel, San Francisco, CA (US); Wenbo Hou, Lancaster, CA (US); Edward Karst, South Pasadena, CA (US); Taraneh G. Farazi, Santa Clara, CA (US)

(73) Assignee: PACESETTER, INC., Sylmar, CA (US)

(21) Appl. No.: 12/882,099

(22) Filed: Sep. 14, 2010

(51) Int. Cl. A61B 5/0295

(52) U.S. Cl. 600/500

(57) ABSTRACT

Implanted systems and methods for monitoring a patient's arterial stiffness are provided. An implanted sensor is used to produce a signal indicative of changes in arterial blood volume for a plurality of beats of the patient's heart. A pulse duration metric is determined for each of a plurality of pulses of the signal, wherein each pulse of the signal corresponds to a beat of the patient's heart. Arterial stiffness is monitored based on the determined pulse duration metric for the plurality of pulses of the signal. This can include monitoring arterial stiffness based on a dispersion of the pulse duration metric and/or an average of the pulse duration metric.

### FIELD OF THE INVENTION

Embodiments of the present invention relate to implantable systems and methods for use therewith, for monitoring arterial stiffness (also known as vascular stiffness).

### BACKGROUND OF THE INVENTION

Arteries can stiffen as a consequence of age and arteriosclerosis, as well as for other reasons. Increased arterial stiffness is often associated with an increased risk of adverse cardiovascular events. Two leading causes of death, myocardial infarction and stroke, are both believed to be a direct consequence of atherosclerosis.

Normal arteries convert pulsatile ejections of blood from the heart at one end into a steady, even flow at the other end. This function is made possible because arteries are compliant and able to expand due to pressure, and because arteries possess the ability to recoil.

Stiffened arteries require a greater amount of force to expand and take up the blood ejected from the heart. The increased force required to move the blood through the stiffened arteries causes the heart to contract harder to accommodate the artery. Over time, the increased load placed on the heart can become a problem.

Measurements of vascular stiffness usually involve an invasive procedure of inserting a pressure catheter into a patient's vasculature. This makes the continuous measurement of arterial stiffness very difficult.

### SUMMARY OF THE INVENTION

Embodiments of the present invention are related to implantable systems and methods for use therewith. Specific embodiments of the present invention relate to implantable systems that include an implantable sensor and methods for use therewith.

Certain embodiments of the present invention relate to methods for monitoring a patient's arterial stiffness that are for use with an implanted system that includes an implanted sensor capable of producing a signal indicative of changes in arterial blood volume for a plurality of beats of the patient's heart. Such an implanted sensor can be, e.g., an implanted photoplethysmography (PPG) sensor that can be used to obtain a photoplethysmography (PPG) signal indicative of changes in arterial blood volume, or an implanted impedance plethysmography (IPG) sensor that can be used to obtain an impedance plethysmography (IPG) signal indicative of changes in arterial blood volume, but is not limited thereto.

More specifically, such embodiments can include using the implanted sensor to produce the signal (e.g., PPG or IPG signal) indicative of changes in arterial blood volume for a plurality of beats of the patient's heart. A pulse duration metric is determined for each of a plurality of pulses of the signal, wherein each pulse of the signal corresponds to a beat of the patient's heart. Arterial stiffness is monitored based on the determined pulse duration metric for the plurality of pulses of the signal. Additionally, an alarm and/or therapy can be triggered based on the monitored arterial stiffness, and/or therapy can be adjusted.

In accordance with specific embodiments, the pulse duration metric for a pulse of the signal that corresponds to a beat can be a duration of the pulse at a percentage of a peak-to-peak amplitude of the pulse for the beat. Such a percentage can be in the range of 30% to 90%. For a more specific example, the pulse duration metric for a pulse of the signal that corresponds to a beat can be a duration of the pulse at 70% of a peak-to-peak amplitude of the pulse for the beat.

In accordance with some embodiments, the arterial stiffness is monitored by determining a measure of dispersion of the determined pulse duration metric, and monitoring arterial stiffness based on the measure of the dispersion. Exemplary measures of dispersion that can be used include standard deviation, normalized standard deviation, interquartile range, range, mean difference, median absolute deviation, average absolute deviation, coefficient of variation, quartile coefficient of dispersion, relative mean difference, variance, and variance-to-mean ratio of the metric. Arterial stiffness is believed to be inversely related to such measures of dispersion. Thus, the lower the determined measure of dispersion the higher the arterial stiffness, and the higher the determined measure of dispersion the lower the arterial stiffness (where a high arterial stiffness means the arteries are less compliant and thus typically less healthy, and a low arterial stiffness means the arteries are more compliant and thus typically more healthy).

In accordance with other embodiments, the arterial stiffness is monitored by determining an average of the pulse duration metric, and monitoring arterial stiffness based on the determined average. Arterial stiffness is believed to be inversely related to the average of the pulse duration metric. Thus, the lower the determined average of the pulse duration metric the higher the arterial stiffness, and the higher the determined average of the pulse duration metric the lower the arterial stiffness.

This description is not intended to be a complete description of, or limit the scope of, the invention. Other features, aspects, and objects of the various embodiments of the present invention can be obtained from a review of the specification, the figures, and the claims.

### BRIEF DESCRIPTION OF THE FIGURES

FIG. 1 is a high level flow diagram that is used to explain details of monitoring arterial stiffness in accordance with certain embodiments of the present invention.

FIG. 2 is a high level flow diagram of an embodiment of the present invention that uses a measure of dispersion of a pulse duration metric to monitor arterial stiffness.

FIG. 3 is a high level flow diagram of an embodiment of the present invention that uses an average of a pulse duration metric to monitor arterial stiffness.

FIG. 4 illustrates an exemplary pulse of a photoplethysmography (PPG) signal corresponding to a beat of a patient's heart, and an exemplary pulse duration metric that can be determined therefrom.

FIG. 5 is a graph of an exemplary pulse duration metric determined over time for a healthy subject with compliant arteries.

FIG. 6 is a graph of an exemplary pulse duration metric over time for an unhealthy subject with stiffer than normal arteries.

FIG. 7A illustrates an exemplary implantable stimulation device that can be used to perform various embodiments of the present invention.

FIGS. 7B and 7C illustrate exemplary implantable monitoring devices that can be used to perform various embodiments of the present invention.

FIG. 8 is a simplified block diagram that illustrates possible components of the implantable devices as shown in FIGS. 7A-7C.

### DETAILED DESCRIPTION OF THE INVENTION

The following description is of the best modes presently contemplated for practicing various embodiments of the present invention. The description is not to be taken in a limiting sense but is made merely for the purpose of describing the general principles of the invention. The scope of the invention should be ascertained with reference to the claims. In the description of the invention that follows, like numerals or reference designators will be used to refer to like steps, parts or elements throughout. In addition, the first digit of a reference number identifies the drawing in which the reference number first appears.

It would be apparent to one of skill in the art reading this description that the various embodiments of the present invention, as described below, may be implemented in many different embodiments of hardware, software, firmware, and/or the entities illustrated in the Figures. Any actual software, firmware and/or hardware described herein is not limiting of the present invention. Thus, the operation and behavior of the embodiments of the present invention will be described with the understanding that modifications and variations of the embodiments are possible, given the level of detail presented herein.

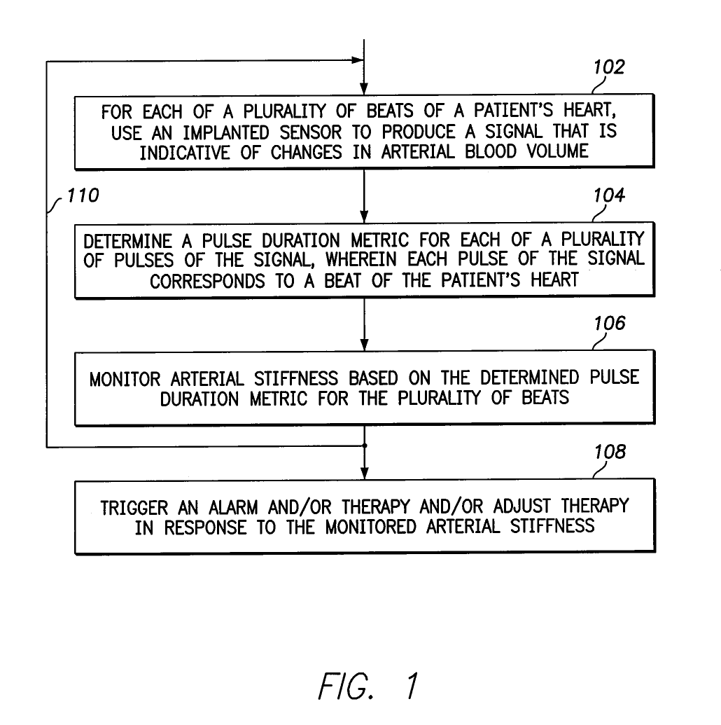

Various embodiments of the present invention for monitoring arterial stiffness will now be summarized beginning with a description of the high level flow diagram of FIG. 1. Where embodiments of the present invention are summarized with reference to the high level flow diagrams, various algorithmic steps are summarized in individual ‘blocks’. Such blocks describe specific actions or decisions that are made or carried out as the algorithm proceeds. Where a microcontroller (or equivalent) is employed, the flow diagrams presented herein provides the basis for a ‘control program’ that may be used by such a microcontroller (or equivalent) to effectuate the desired control of the implantable system. Those skilled in the art may readily write such a control program based on the flow diagram and other descriptions presented herein. Embodiments of the present invention are not limited to the exact order and/or boundaries of the steps shown in the flow diagrams. In fact, many of the steps can be performed in a different order than shown, and many steps can be combined, or separated into multiple steps. All such variations are encompassed by the present invention. The only time order is important is where a step acts on the result of a previous step.

In step 102, for each of a plurality of beats of a patient's heart, an implanted sensor is used to produce a signal that is indicative of changes in arterial blood volume. The sensor can be, e.g., an implantable photoplethysmography (PPG) sensor that is used to produce a photoplethysmography (PPG) signal, an implantable impedance plethysmography (IPG) sensor that is used to produce an impedance plethysmography (IPG) signal, or some other type of sensor, examples of which are provided below.

A normal blood pressure signal is a sum of a forward moving pressure wave (i.e., an incident wave) and a reflected wave. A characteristic notch on the normal blood pressure signal may indicate the point at which these two waves collide. The normal blood pressure signal morphology varies with respect to stiffness of the artery, and the velocity of the travelling waves is proportional to the stiffness of the artery. For instance, in a stiff blood vessel, the notch occurs closer to the peak of the normal blood pressure waveform as the reflected wave comes back sooner than in a non-stiff vessel.

The morphology of signals indicative of changes in arterial blood volume, such as PPG and IPG signals, resemble the morphology of a normal blood pressure signal obtained from a pressure measuring device. Features can thus be extracted from a signal indicative of changes in arterial blood pressure to monitor a level of arterial stiffness, as will be explained below.

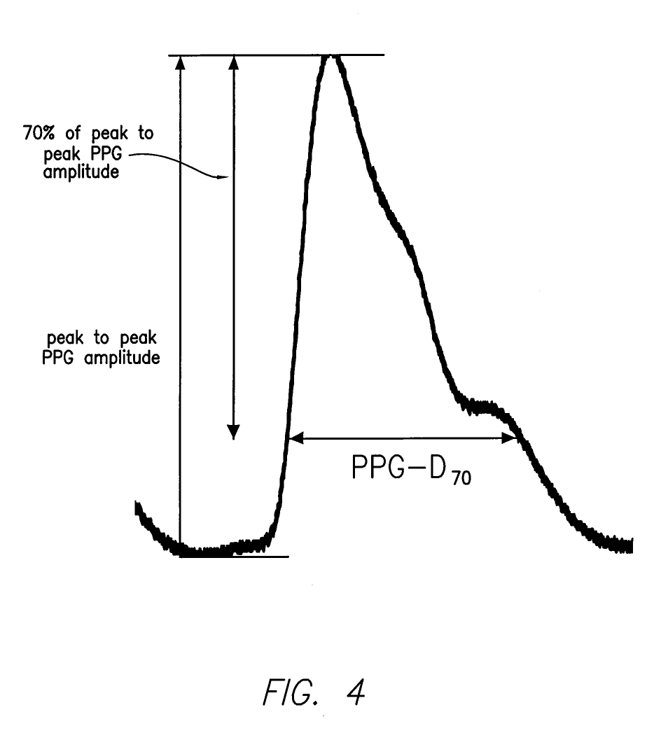

In step 104, for each of a plurality of pulses of the signal indicative of changes of arterial blood volume, a pulse duration metric is determined. The pulse duration metric can be any metric that is related to the length or duration of a pulse in the signal. An exemplary pulse duration metric is the duration of a pulse of the signal at a percentage of a peak-to-peak amplitude of the pulse. In specific embodiments, the percentage is in the range of 30% to 90%. In FIG. 4, the pulse duration metric is the duration of the PPG signal at 70% of the peak-to-peak amplitude, with such metric being labeled using the short-hand annotation “PPG-D₇₀”. Where the pulse duration metric is at 50% of the peak-to-peak amplitude, the pulse duration metric is also known as the Full Width at Half Maximum (FWHM). Other examples of a pulse duration metric that can be used at step 104 include, but are not limited to, times between features of the pulse, such as time between a feature on the upward slope of the pulse and a feature on the downward slope of the pulse. For example, the time from foot to foot, or the time from the maximum upward slope to the maximum downward slope can be used as a pulse duration metric. For another example, the time from a foot to the following peak, or from the peak to the following foot, can be used as a pulse duration metric. In accordance with an embodiment, the signal indicative of changes of arterial blood volume (e.g., the PPG signal) can be normalized before the pulse duration metrics are determined, or alternatively, after the pulse duration metrics are measured the metrics can be normalized with respect to the amplitude the signal indicative of changes of arterial blood volume (e.g., the amplitude of the PPG signal).

Pulse duration metrics are inversely proportional to the stiffness of the arteries in the pathway from the sensor site to the resistance vessels or bifurcations that create the reflected wave. The stiffer the arteries, the sooner the reflected wave returns, leading to narrower pulses. As will be appreciated by the discussion herein, certain embodiments of the present invention take advantage of this phenomenon.

Furthermore, when an arterial path has greater distensibility, there is greater variation in transit times for the forward-travelling and reverse-travelling pressure waves. For instance, if arteries are highly compliant, there may be substantial beat-to-beat variations in the pressure pulsations, leaving a less uniform blood distribution pattern on individual beats. However, if arteries are stiff, the blood flow pulses are not only more narrow but also more consistent. As will be appreciated by the discussion herein, certain embodiments of the present invention take advantage of this phenomenon.

In accordance with specific embodiments, the pulse duration metric can be determined for pulses that corresponds to beats within a pre-determined window or number of beats. In certain embodiments, the number of beats is preferably at least 20 in order to capture at least one respiration cycle, so that a respiratory effect on the pulse duration metric can be substantially cancelled.

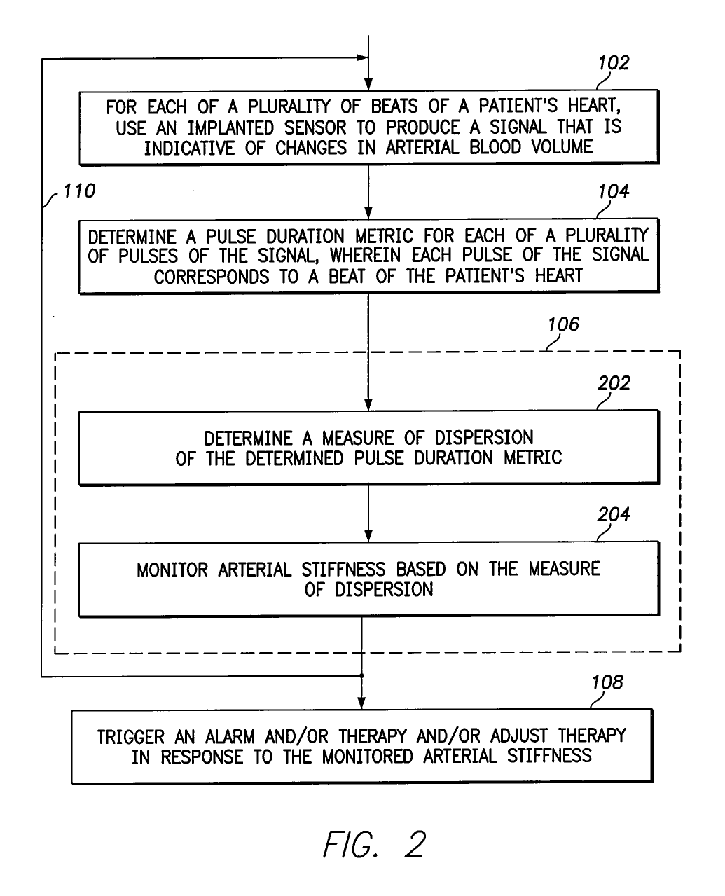

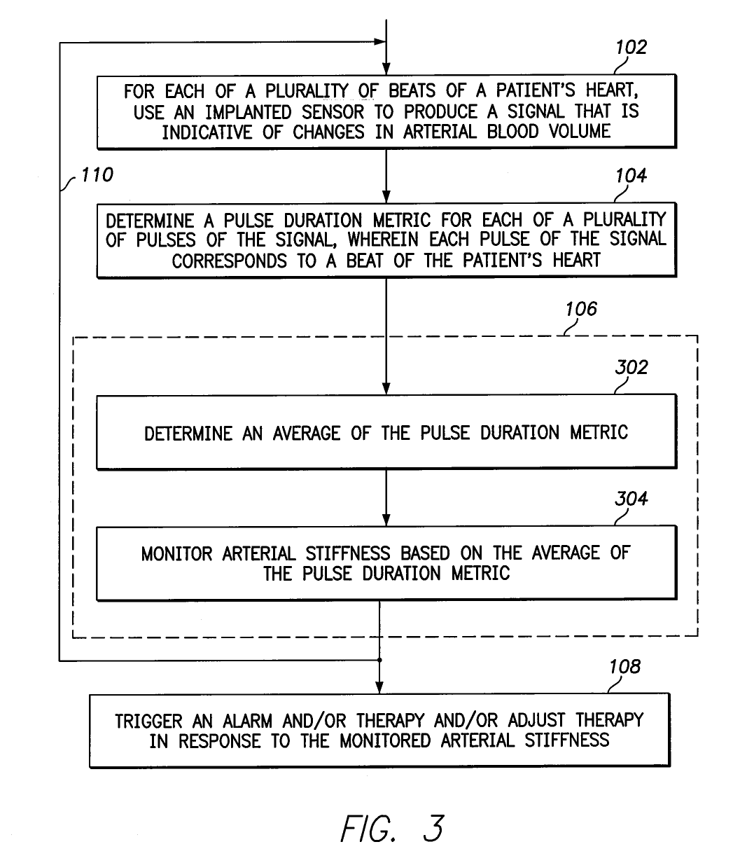

Still referring to FIG. 1, in step 106, the arterial stiffness is monitored based on the determined pulse duration metric for the plurality of pulses of the signal indicative of changes in arterial blood volume. As discussed below with respect to FIGS. 2 and 3, a measure associated with arterial stiffness, such as the dispersion or average of the pulse duration metric, can be determined from the pulse duration metric and used for the monitoring. The measure can be compared to a threshold or a trend over time can be monitored, as will be described in more detail below.

As indicated by line 110, steps 102, 104 and 106 can be repeated from time to time, e.g., periodically, or in response to a triggering event. For example, steps 102, 104 and 106 can be performed substantially continually, or periodically (e.g., once an hour, a day, a week, or the like). Alternatively, steps 102, 104 and 106 can be performed aperiodically, e.g., in response to a triggering event. The repeating of these steps enables changes in arterial stiffness to be monitored, so that increases or decreases (or substantially no changes) in arterial stiffness over time can be detected.

As indicated at step 108, an alarm and/or therapy can be triggered, and/or therapy can be adjusted in response to the monitored arterial stiffness. Step 108 can also include storing information and/or analyzing information in alternative manners. For example, in accordance with specific embodiments of the present invention, a pulse duration metric, a measure of dispersion of pulse duration metric, an average of a pulse duration metric and/or some other measure of a patient's arterial stiffness, and potentially other information is stored within memory of the implantable system for later analysis within the device and/or for later transmission to an external device. Such an external device (e.g., an external programmer or external monitor) can then be used to analyze such data.

As mentioned above, the signal indicative of changes in arterial blood volume can be a PPG signal. Volume changes in blood vessels occur in a pulsatile manner with each beat of the heart as blood flows in and out of a portion of the body. A PPG sensor produces waveform measurements reflecting changes in arterial blood volume. These waveforms measurements are similar to arterial pressure waveform measurements because changes in arterial pressure correspond to relative changes in arterial blood volume.

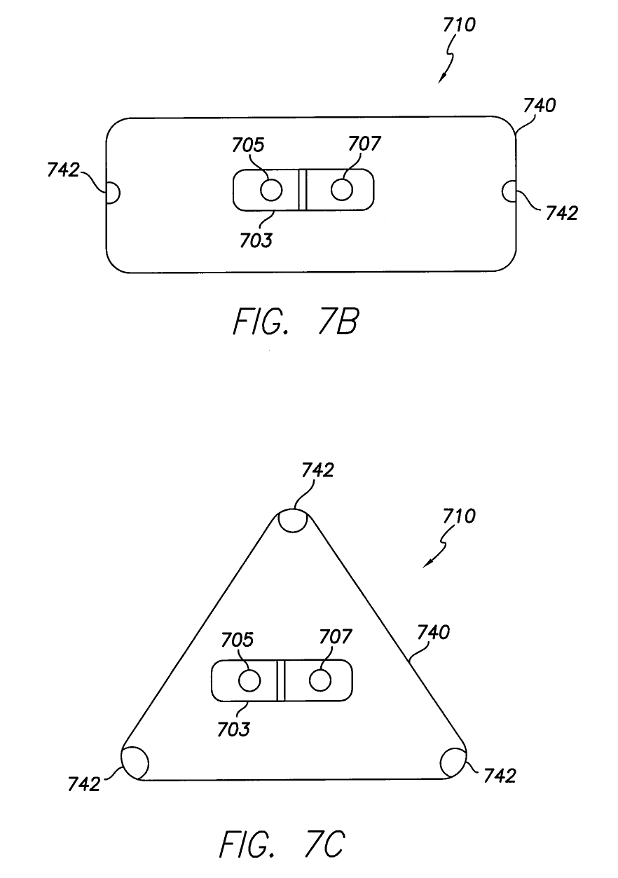

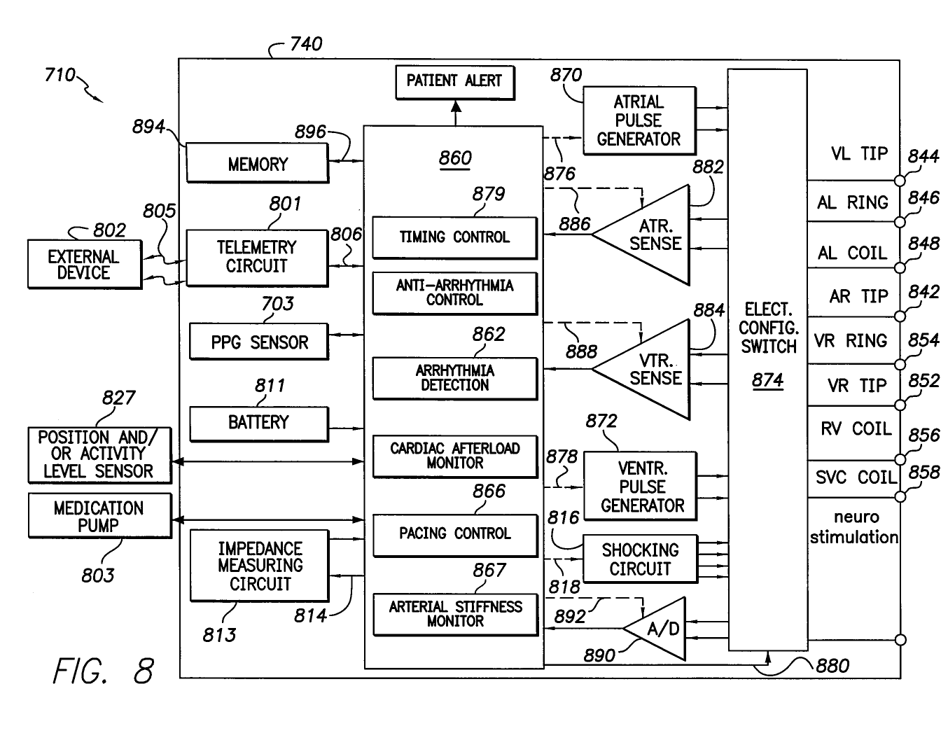

Exemplary PPG sensors are discussed below with reference to FIGS. 7A-7C. The PPG sensor can be implanted, e.g., in the pectoral region of a patient. Thus, it is practical that the PPG sensor can be integrated with or attached to the housing of a pacemaker or Implantable Cardioverter-Defibrillator (ICD), as can be appreciated from FIGS. 7A and 8, as discussed below. Alternative locations for implantation of the PPG sensor include, but are not limited to, the patient's abdomen.

As mentioned above, the signal indicative of changes in arterial blood volume can be an IPG signal. An impedance plethysmography sensor can measure changes in blood volume for a specific body segment. As the arterial blood volume changes, the electrical resistance also changes.

The IPG sensor can be implanted, e.g., in the pectoral region of a patient. Thus, it is practical that the IPG sensor can be integrated with or attached to the housing of a pacemaker or ICD, as can be appreciated from FIGS. 7A and 8 as discussed below. Alternative locations for implantation of the IPG sensor include, but are not limited to, the patient's abdomen.

In still other embodiments, the signal indicative of changes in arterial blood volume can be other types of signals produced using other types of sensors. For example, the signal indicative of changes in arterial blood volume can be a signal output by a sensor including a piezo-electric diaphragm. Alternative sensors that can be used to produce the signal indicative of changes in arterial blood volume, include, but are not limited to, a close range microphone, a sensor including a small mass on the end of a piezo bending beam with the mass located on the surface of a small artery, a transmission mode infrared motion sensor sensing across the surface of a small artery, or a micro-electro-mechanical systems (MEMS) accelerometer located on the surface of a small artery. Such alternative sensors can be located, e.g., on the tip of a short lead connected to a device that is subcutaneously implanted. The implanted sensor can be extra vascular, and a sufficient distance from the patient's heart such that meaningful changes in the amount of time it takes a pulse wave originating in the heart to reach the implanted sensor can be detected, thereby enabling changes in arterial blood pressure to be detected. For example, the implanted sensor (used to obtain the signal indicative of changes in arterial blood volume) can be at least 10 mm from the patient's aortic root. Such a sensor can be implanted, e.g., in the pectoral region of a patient. An alternative location for implantation of the sensor includes, but is not limited to, the patient's abdominal region.

Referring now to FIG. 2, step 106 is shown as including steps 202 and 204, in accordance with an embodiment of the present invention. In step 202, a measure of dispersion of the pulse duration metric is determined based on the results of step 104. This measure of dispersion can be a standard deviation, normalized standard deviation, interquartile range, range, mean difference, median absolute deviation, and average absolute deviation, coefficient of variation, quartile coefficient of dispersion, relative mean difference, variance, variance-to-mean ratio of the metric or any other measure of dispersion. In step 204, arterial stiffness is monitored based on the measure of the dispersion. Arterial stiffness is believed to be inversely related to such measures of dispersion. Thus, the lower the determined measure of dispersion the higher the arterial stiffness, and the higher the determined measure of dispersion the lower the arterial stiffness (where a high arterial stiffness means the arteries are less compliant and thus typically less healthy, and a low arterial stiffness means the arteries are more compliant and thus typically more healthy).

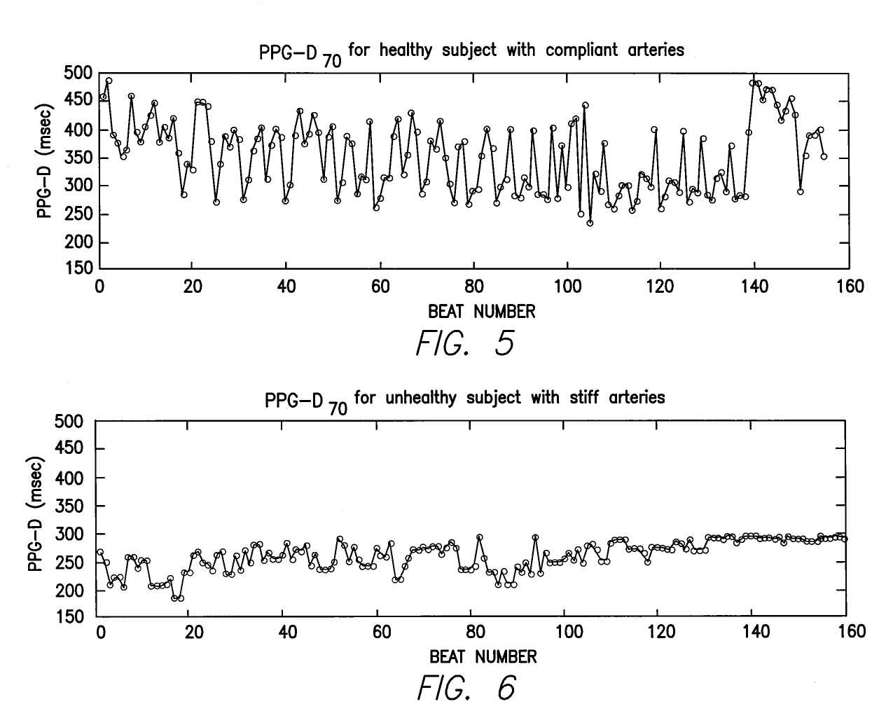

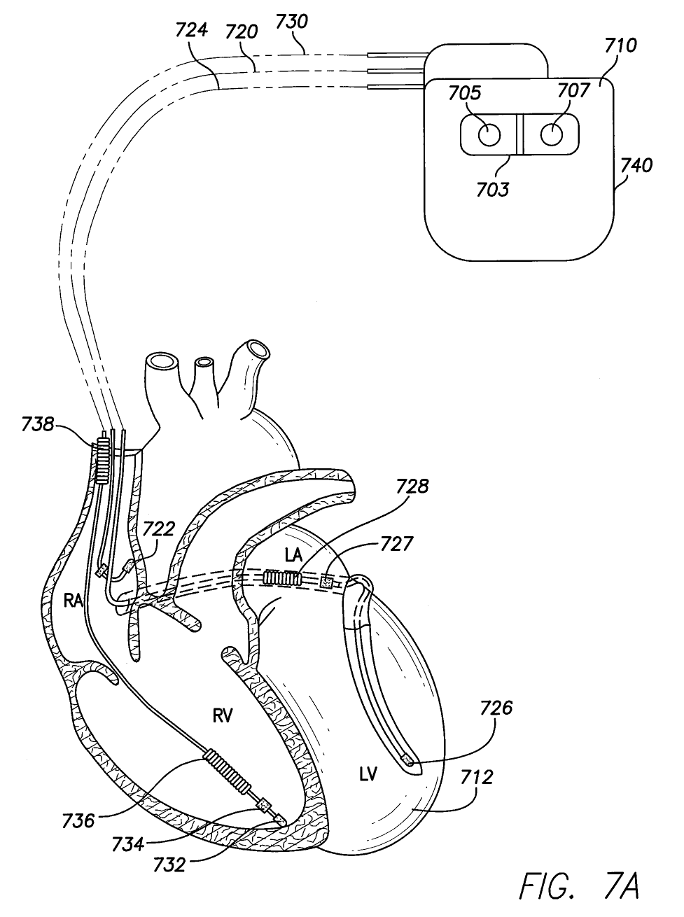

An example of the use of a measure of dispersion to monitor arterial stiffness is shown with respect to FIGS. 5 and 6. FIG. 5 shows the beat-to-beat PPG-D₇₀ (i.e., the duration of the PPG signal at 70% of the peak-to-peak amplitude) for beats of a young healthy subject, whereas FIG. 6 shows the beat-to-beat PPG-D₇₀ for beats of a subject who is known to have stiff arteries. The standard deviation (which is an example of a measure of dispersion) of PPG-D₇₀ over 160 beats for the young healthy subject represented in FIG. 5 is 62.3 msec, whereas the standard deviation of PPG-D₇₀ for the subject represented in FIG. 6 over 290 beats is 23 msec. The percent normalized standard deviation over the average PPG-D (percent normalized=100%×standard deviation/average) is 18% for the subject represented in FIG. 5 and 8.5% for the subject represented in FIG. 6. In this example, the healthy young subject demonstrated a percent normalized standard deviation which is over twice that of the subject whose arteries were known to be stiff. Thus, FIGS. 5 and 6 illustrate that a measure of dispersion, such as normalized standard deviation, appears to be a good indicator of arterial stiffness.

Step 204 can include, e.g., a determination of whether the measure of dispersion is below a specified threshold. If so, abnormal (e.g., higher than normal) arterial stiffness may be detected. Additionally or alternatively, trend data in the measure of dispersion over multiple windows can be monitored to detect changes in arterial stiffness over time. In other words, by repeating steps 102, 104, 202 and 204 from time to time (as indicated by line 110), changes in arterial stiffness can be monitored. More specifically, decreases in the measure of dispersion over time are indicative of increases in arterial stiffness (and thus, decreases in arterial compliance), and increases in the measure of dispersion over time are indicative of decreases in arterial stiffness (and thus, increases in arterial compliance).

Referring now to FIG. 3, step 106 is shown as including steps 302 and 304, in accordance with an embodiment of the present invention. In step 302, an average of the pulse duration metric is determined based on the results of step 104. In step 304, arterial stiffness is monitored based on the average. Arterial stiffness is believed to be inversely related to the average of the pulse duration metric. Thus, the lower the determined average of the pulse duration metric the higher the arterial stiffness, and the higher the determined average of the pulse duration metric the lower the arterial stiffness.

Step 304 can include, e.g., a determination of whether the average of the pulse duration metric is below a specified threshold. If so, abnormal (e.g., higher than normal) arterial stiffness may be detected. Additionally or alternatively, trend data in the average of the pulse duration metric over multiple windows can be monitored to detect changes in arterial stiffness over time. In other words, by repeating steps 102, 104, 302 and 304 from time to time (as indicated by line 110), changes in arterial stiffness can be monitored. More specifically, decreases in the average of the pulse duration metric over time are indicative of increases in arterial stiffness (and thus, decreases in arterial compliance), and increases in the average of the pulse duration metric over time are indicative of decreases in arterial stiffness (and thus, increases in arterial compliance).

The embodiments described with reference to FIGS. 2 and 3 can also be combined such that arterial stiffness is monitored based on both the dispersion of the pulse duration metric (and/or changes therein) and the average of the pulse duration metric (and/or changes therein).

Exemplary details of step 108 (in FIGS. 1-3) will now be discussed. In accordance with specific embodiments of the present invention, an alarm can be triggered at step 108 based on comparisons of the measure of dispersion of pulse duration metric (and/or the changes therein), the average of the pulse duration metric (and/or the changes therein) and/or some other measure indicative of the patient's arterial stiffness, to one or more corresponding threshold(s). For example, if the dispersion of the pulse duration metric decreases by a specified threshold, an alarm can be triggered. Such an alarm can be part of an implanted system. Alternatively, an implanted system can trigger a non-implanted alarm of a non-implanted system. In still other embodiments, where such information is transmitted (e.g., via telemetry) to an external device, a non-implanted alarm can be triggered based on comparisons of the measure of dispersion of pulse duration metric, the average of the pulse duration metric and/or some other indicator of the patient's arterial stiffness, to one or more corresponding threshold(s).

A decrease in a measure of dispersion or average of the pulse duration metrics corresponds to an increase in arterial stiffness. In one embodiment, a sufficiently large increase in arterial stiffness will warrant therapy and/or an alarm. In certain embodiments, triggered therapy can involve adjusting at least one pacing parameter of a cardiac stimulation device based on a measure indicative of arterial stiffness or based on a trend in such a measure. Exemplary pacing parameters that can be adjusted include AV delay, V to V timing in a cardiac resynchronization therapy (CRT) device, as well as base rate, or electrode configuration and pace timing in a multi-electrode lead, but are not limited hereto. In specific embodiments, a set of pacing parameters can be selected to produce a more elastic artery. For example, the heart rate can be slowed down in a rest mode to induce an elastic artery.

In certain embodiments, medicine, such as statins, can be supplied based on the determined measure indicative of arterial stiffness or a change in detected measure indicative of arterial stiffness. For example, medicine can be provided from an implanted medicine pump or externally based on the monitored arterial stiffness or a change therein.

In certain embodiments, the trending of the measure indicative of arterial stiffness can be used to guide cardiovascular therapy, such as a recommended dosage of statins.

As mentioned above, various steps described with reference to the flow diagrams can be repeated from time-to-time, to thereby track changes in the various measures indicative of a patient's arterial stiffness. For example, steps of a flow diagram can be performed periodically (e.g., once a minute, hour, day, week, or the like). The measures indicative of a patient's arterial stiffness can be compared in real time to corresponding thresholds. Alternatively, or additionally, measures indicative of a patient's arterial stiffness can be stored in memory of the implanted system. Such stored values can be analyzed by the implanted system and/or transmitted (e.g., via telemetry) to an external system (e.g., external programmer and external monitor) and analyzed by the external system. Use of various thresholds can be used to trigger alarms and/or therapy, as will be described below. In accordance with an embodiment of the present invention, information related to periods when the measure of dispersion falls below a threshold (indicative of an abnormal arterial stiffness) can be stored. This can include, for example, determining and storing timing and duration information indicative of how long the threshold is crossed during a period of time (e.g., per day, or per week) in order to provide a measure of arterial stiffness burden. Such information can be displayed with previously determined arterial stiffness burdens (e.g., from a month ago) and compared to see improvement or worsening of arterial stiffness burden. Such information can be continually, or from time to time, automatically uploaded to an external device. Such an external monitoring device can be located, e.g., in the patients' home, and the information can be transmitted (e.g., through telephone lines or the Internet) to a medical facility where a physician can analyze the information. Alternatively, the external device can be located at a medical facility, and the information can be uploaded when the patient visits the facility.

Depending on the frequency, periodic monitoring of a patient's arterial stiffness may be costly in terms of energy, memory and/or processing resources. Accordingly, it may be more efficient to trigger the performance of certain steps upon detection of an event, such as a specific activity, or lack thereof, and/or a specific posture of the patient. For example, an activity sensor and/or posture sensor can be used to trigger the performance of steps of a flow diagram. For example, steps can be triggered when it is detected that a patient is inactive and lying down. Additionally, or alternatively, steps can be triggered when a patient is upright and walking. In still other embodiments, steps can be triggered to occur, at specific intervals following a patient changing their posture (e.g., assuming an upright posture) and/or activity level. For example, following a triggering event, a measure indicative of a patient's arterial stiffness can be determined once a minute for 10 minutes, or at 1 minute, 2 minutes, 5 minutes and 10 minutes after the triggering event. Of course, other variations are also possible, and within the scope of the present invention. It may also be that one or more specific step is/are performed substantially continually, but other steps are only performed in response to a triggering event, such as those discussed above.

In specific embodiments, a patient's position and/or activity level is determined using one or more position and/or activity level sensors, such as the position and/or activity level sensor 827 shown in FIG. 8. The sensor can be, e.g., a DC-coupled 3-dimensional accelerometer as described in U.S. Pat. No. 6,658,292 (Kroll et al), a multi-axis DC accelerometer as described in U.S. Pat. No. 6,466,821 (Pianca et al), or an external field sensor as described in U.S. Pat. No. 6,625,493 (Kroll et al), each of which are incorporated herein by reference. Such sensors are able to distinguish among different static positions. In addition, since the sensors can detect motion, they can be used to distinguish between a static vertical position, such as sitting, and a standing position, which due to the dynamics of balance is associated with subtle motion that is not present while sitting. In this way an implantable system, using one of the above mentioned sensors or other sensing modality, can detect a change in body position (i.e., posture), which can be used as a trigger to perform specific methods of the present invention described below.

The morphology of the signal indicative of changes in arterial blood volume (produced at step 102) may be effected by a patient's posture and/or activity level. Thus, it can be useful to only determine measures indicative of a patient's arterial stiffness during a specific posture and/or activity level. In still other embodiments, measures indicative of a patient's arterial stiffness can be determined regardless of the patient's posture and/or activity level, but correlated to the patient's posture and/or activity level. For example, where at least some of steps are triggered in response to detection of various different activity and/or posture states, information about the patient's activity and/or posture can also be stored along with the measures indicative of a patient's arterial stiffness, so that such information can be correlated. In other words, there could be a cross-correlation of measures indicative of a patient's arterial stiffness with levels of activity and/or posture.

As mentioned above, the determination of the pulse duration metric can be triggered based on the position and/or activity level. For example, the collection of the pulse width duration metrics can be triggered based on, or associated with, the position and/or activity level to ensure that the pulse width duration metrics and/or measures derived from the pulse width duration metrics correspond to the same position and/or activity level.

As mentioned above, the measure indicative of arterial stiffness or change in the measure indicative of arterial stiffness can be determined using the pulse duration metrics and the activity level and/or position. In one embodiment, the measure indicative of arterial stiffness, such as a measure of dispersion or average of a pulse duration metric, can be determined for pulses corresponding of the same activity level and/or position. The measure can be compared to past measures at the same or similar activity level and/or position. Alternately, the measure can be compared to a specific threshold associated with the activity level and/or position. In this way, differences in the measure indicative of arterial stiffness caused by different positions and/or activity levels can be accounted for.

In one embodiment, positions and/or activity levels that promote the least amount of arterial stiffness can be determined, and these positions and/or activity levels can be recommended to the user. Further, any therapy and/or alarm discussed above can be appropriate to the position and/or activity level. For example, in one embodiment, therapy is provided and/or an alarm produced only when the user is resting and/or supine.

Other types of measures of physiological conditions, such as heartbeat or other physiological data, may be used along with the present invention. For example, in a specific embodiment, a measure indicative of arterial stiffness is only determined when the patient's heart rate is within a specified range or below a threshold. More generally, in certain embodiments, measures indicative of arterial stiffness are only determined during consistent physiological conditions. In other embodiments, arterial stiffness measurements can be taken at any time, regardless of activity or other physiologic conditions of the patient.

In the above described embodiments, the determination of a measure indicative of arterial stiffness can be triggered by the implantable device, e.g., after a certain amount of time occurred since a last measurement. In certain embodiments, even after the specified amount of time occurred since the last measurement, a measure indicative of arterial stiffness may not be determined until the patient is determined to be at rest, or until other physiological conditions occur. It is also possible that a measure indicative of arterial stiffness is triggered telemetrically by an external device.

Photoplethysmography (PPG) and impedance plethysmography (IPG) signals (collectively referred to as PPG/IPG signals), and other plethysmography signals, show changes in a patient's arterial system as a result of the patient's heart contracting, and such signals are indicative of changes in arterial blood volume. A PPG signal can be obtained using a PPG sensor, which can be an optical sensor including a light source and a light detector. An IPG signal can be obtained using an IPG sensor, which can include electrodes and circuitry used to measure the impedance between such electrodes. One or more such electrodes can be located on one or more leads, and/or a mechanical housing of an implanted device can act as one of the electrodes. Other types of plethysmography signals indicative of changes in arterial blood volume can be obtained using other types of sensors, as described above.

In some embodiments, monitoring of arterial stiffness can be improved if the PPG/IPG signals (or other plethysmography signals) used in the above described embodiments are appropriately processed. For example, recording of a plethysmography signal may be triggered, e.g., on an R wave, based on respiratory cycle, based on activity levels, etc. The plethysmography signal can be filtered to remove respiratory noise, motion artifacts, baseline drift, etc. For example, the signal can be band-pass filtered so that the pass-band is from about 0.7 to 10 Hz, although other pass bands can be used. Most of the respiration signal and high frequency noise can be removed by such filtering.

Additionally, an outlier removal process can be performed to remove “bad” heart beats. For example, the outlier removal can be accomplished by grouping a plurality of (e.g., 20) consecutive heart beats, determining a mean of the filtered plethysmography signal for the plurality of heart beats, and then comparing the determined mean to individual cycles of the filtered plethysmography signal. Further, outlier removal can be performed by removing each cardiac cycle of the filtered plethysmography signal that deviates by at least a threshold amount (e.g., 3 or some other number of standard deviations) from the mean of the plethysmography signal for the plurality of consecutive beats. The cycles of the plethysmography signal remaining after the outlier removal step can then be ensemble averaged, with the result being an average representation of the plethysmography signal for the plurality of consecutive beats, with noise and “bad” beats removed.

Thereafter, features of the plethysmography signal can be detected from the ensemble-averaged plethysmography signal and/or metrics can be determined from the ensemble-averaged plethysmography signal.

### Exemplary Implantable System

FIGS. 7A-7C and 8 will now be used to describe exemplary implantable systems that can be used to monitor arterial stiffness, in accordance with embodiments of the present invention. Referring to FIG. 7A, the implantable system is shown as including an implantable stimulation device 710, which can be a pacing device and/or an implantable cardioverter defibrillator. The device 710 is shown as being in electrical communication with a patient's heart 712 by way of three leads, 720, 724 and 730, which can be suitable for delivering multi-chamber stimulation and shock therapy. The leads can also be used to obtain IEGM signals, for use in embodiments of the present invention. Instead of having leads with electrodes attached to the heart, it is also possible that subcutaneous electrodes can be used to obtain ECG signals. In still other embodiments, it is possible that the electrodes are located on the housing of the implantable device 710, and that such electrodes are used to obtain subcutaneous ECG signals. In this latter embodiment, the device 710 may not be capable of pacing and/or defibrillation, but rather, the implantable device 710 can be primarily for monitoring purposes.

The implantable system is also shown as including an implantable photoplethysmography (PPG) sensor 703 that can be used to produce a PPG signal, a cycle of which can be similar to the waveform shown in FIG. 4. Referring to FIG. 7A, the PPG 703 sensor includes a light source 705 and a light detector 707. The light source 705 can include, e.g., at least one light-emitting diode (LED), incandescent lamp or laser diode. The light detector 707 can include, e.g., at least one photoresistor, photodiode, phototransistor, photodarlington or avalanche photodiode. Light detectors are often also referred to as photodetectors or photocells.

The light source 705 outputs light that is reflected or backscattered by surrounding patient tissue, and reflected/backscattered light is received by the light detector 707. In this manner, changes in reflected light intensity are detected by the light detector, which outputs a signal indicative of the changes in detected light. The output of the light detector can be filtered and amplified. The signal can also be converted to a digital signal using an analog to digital converter, if the PPG signal is to be analyzed in the digital domain. Additional details of exemplary implantable PPG sensors are disclosed in U.S. Pat. Nos. 6,409,675 and 6,491,639, both to Turcott and both entitled “Extravascular Hemodynamic Sensor”, which are incorporated herein by reference.

A PPG sensor can use a single wavelength of light, or a broad spectrum of many wavelengths. In the alternate embodiments, the light source can be any source of radiant energy, including laser diode, heated filament, and ultrasound transducer. The detector can be any detector of radiant energy, including phototransistor, photodetector, ultrasound transducer, piezoelectric material, and thermoelectric material.

It is generally the output of the photodetector that is used to produce a PPG signal. However, there exists techniques where the output of the photodetector is maintained relatively constant by modulating the drive signal used to drive the light source, in which case the PPG signal is produced using the drive signal, as explained in U.S. Pat. No. 6,731,967, to Turcott, entitled “Methods and Devices for Vascular Plethysmography via Modulation of Source Intensity,” which is incorporated herein by reference.

The PPG sensor 703 can be attached to a housing 740 of an implantable device, which as mentioned above can be, e.g., a pacemaker and/or an implantable cardioverter-defibrillator (ICD), or a simple monitoring device. Exemplary details of how to attach a sensor module to an implantable cardiac stimulation device are described in U.S. Pat. No. 7,653,434, to Turcott et al., entitled “Autonomous Sensor Modules for Patient Monitoring”, which is incorporated herein by reference. It is also possible that the PPG sensor 703 be integrally part of the implantable cardiac stimulation device 710. For example, the PPG sensor 703 can be located within the housing 740 of an ICD (or pacemaker) that has a window through which light can be transmitted and detected. In a specific embodiment, the PPG sensor 703 has a titanium frame with a light transparent quartz window that can be welded into a corresponding slot cut in the housing of the ICD. This will insure that the ICD enclosure with the welded PPG sensor will maintain a hermetic condition. In alternative embodiments, the PPG sensor can be remote from housing 740 and can communicate with components within the housing via a bus (e.g., including one or more wires), or wirelessly, but is not limited thereto.

Where the PPG sensor 703 is incorporated into or attached to a chronically implantable device 710, the light source 705 and the light detector 707 can be mounted adjacent to one another on the housing or header of the implantable device. The light source 705 and the light detector 707 are preferably placed on the side of the implantable device 710 that, following implantation, faces the chest wall, and are configured such that light cannot pass directly from the source to the detector. The placement on the side of the device 710 that faces the chest wall maximizes the signal to noise ratio by directing the signal toward the highly vascularized musculature, and shielding the source and detector from ambient light that enters the body through the skin. Alternatively, at the risk of increasing susceptibility to ambient light, the light source 705 and the light detector 707 can be placed on the face of the device 710 that faces the skin of the patient.

The implantable PPG sensor 703 outputs a PPG signal a cycle of which can be similar to waveform shown in FIG. 4. Such a signal can be filtered and/or amplified as appropriate, e.g., to remove respiratory affects on the signal, and the like. Additionally, the signal can be digitized using an analog to digital converter. Based on the PPG signal the pulse duration metrics can be determined.

Still referring to FIG. 7A, to sense atrial cardiac signals and to provide right atrial chamber stimulation therapy, the device 710 is coupled to an implantable right atrial lead 720 having at least an atrial tip electrode 722, which typically is implanted in the patient's right atrial appendage. To sense left atrial and ventricular cardiac signals and to provide left-chamber pacing therapy, the device 710 is coupled to a “coronary sinus” lead 724 designed for placement in the “coronary sinus region” via the coronary sinus for positioning a distal electrode adjacent to the left ventricle and/or additional electrode(s) adjacent to the left atrium. As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus.

Accordingly, an exemplary coronary sinus lead 724 is designed to receive atrial and ventricular cardiac signals and to deliver left ventricular pacing therapy using at least a left ventricular tip electrode 726, left atrial pacing therapy using at least a left atrial ring electrode 727, and shocking therapy using at least a left atrial coil electrode 728.

The device 710 is also shown in electrical communication with the patient's heart 712 by way of an implantable right ventricular lead 730 having, in this embodiment, a right ventricular tip electrode 732, a right ventricular ring electrode 734, a right ventricular (RV) coil electrode 736, and an SVC coil electrode 738. Typically, the right ventricular lead 730 is transvenously inserted into the heart 712 so as to place the right ventricular tip electrode 732 in the right ventricular apex so that the RV coil electrode 736 will be positioned in the right ventricle and the SVC coil electrode 738 will be positioned in the superior vena cava. Accordingly, the right ventricular lead 730 is capable of receiving cardiac signals and delivering stimulation in the form of pacing and shock therapy to the right ventricle.

FIG. 7B illustrates an alternative embodiment of the implantable device 710. Here the housing 740 of the device is shown as small, thin, and oblong, with smooth surfaces and a physiologic contour which minimizes tissue trauma and inflammation. The oblong geometry of the housing 740 is desirable because it maximizes separation of electrodes 742 and prevents rotation of the monitor within the tissue pocket, thereby allowing interpretation of morphology features in an ECG sensed using electrodes 742. Two ECG electrodes 742 are shown, however more can be present. In the alternate embodiment illustrated in FIG. 7C, three ECG electrodes 742 are present, one at each apex of the triangle formed by the device housing 740. These three electrodes allow the three standard surfaces ECG leads I-III to be approximated. In an embodiment, four or more ECG electrodes might be used, with each orthogonal electrode pair providing orthogonal ECG signals. Alternatively, an embodiment lacking ECG electrodes is possible. A further alternative has a single ECG electrode with the monitor housing acting as the other electrode in the pair. U.S. Pat. No. 6,409,675, which was incorporated above by reference, provides some additional details of an implantable monitor that includes ECG electrodes on its housing and a PPG sensor. FIGS. 7B and 7C show that the implantable device 710 also includes a PPG sensor 703.

FIG. 8 will now be used to provide some exemplary details of the components of the implantable devices 710. The implantable device 710 can include logic to determine and monitor arterial stiffness.

Referring now to FIG. 8, each of the above implantable devices 710, and alternative versions thereof, can include a microcontroller 860. As is well known in the art, the microcontroller 860 typically includes a microprocessor, or equivalent control circuitry, and can further include RAM or ROM memory, logic and timing circuitry, state machine circuitry, and I/O circuitry. Typically, the microcontroller 860 includes the ability to process or monitor input signals (data) as controlled by a program code stored in a designated block of memory. The details of the design of the microcontroller 860 are not critical to the present invention. Rather, any suitable microcontroller 860 can be used to carry out the functions described herein. The use of microprocessor-based control circuits for performing timing and data analysis functions are well known in the art. In specific embodiments of the present invention, the microcontroller 860 performs some or all of the steps associated with monitoring arterial stiffness. Additionally, the microcontroller 860 may detect arrhythmias, and select and control delivery of anti-arrhythmia therapy.

Representative types of control circuitry that may be used with the invention include the microprocessor-based control system of U.S. Pat. No. 4,940,052 to Mann et al., entitled “Microprocessor Controlled Rate-Responsive Pacemaker Having Automatic Rate Response Threshold Adjustment”, and the state-machines of U.S. Pat. No. 4,712,555, to Thornander, et al., entitled “Physiologically Responsive Pacemaker and Method of Adjusting the Pacing Interval Thereof”; and U.S. Pat. No. 4,944,298 to Sholder, entitled “Atrial Rate Based Programmable Pacemaker with Automatic Mode Switching Means”. For a more detailed description of the various timing intervals used within the pacing device and their inter-relationship, see U.S. Pat. No. 4,788,980 to Mann et al., entitled “Pacemaker Having PVC Response and PMT Terminating Features”. The '052, '555, '298 and '980 patents are incorporated herein by reference.

Depending on implementation, the device 710 can be capable of treating both fast and slow arrhythmias with stimulation therapy, including pacing, cardioversion and defibrillation stimulation. While a particular multi-chamber device is shown, this is for illustration purposes only, and one of skill in the art could readily duplicate, eliminate or disable the appropriate circuitry in any desired combination to provide a device capable of treating the appropriate chamber(s) with pacing, cardioversion and defibrillation stimulation. For example, where the implantable device is a monitor that does not provide any therapy, it is clear that many of the blocks shown may be eliminated.

The housing 740, shown schematically in FIG. 8, is often referred to as the “can”, “case” or “case electrode” and may be programmably selected to act as the return electrode for all “unipolar” modes. The housing 740 may further be used as a return electrode alone or in combination with one or more of the coil electrodes, 728, 736 and 738, for shocking purposes. The housing 740 can further include a connector (not shown) having a plurality of terminals, 842, 844, 846, 848, 852, 854, 856, and 858 (shown schematically and, for convenience, the names of the electrodes to which they are connected are shown next to the terminals). As such, to achieve right atrial sensing and pacing, the connector includes at least a right atrial tip terminal (A_{R} TIP) 842 adapted for connection to the atrial tip electrode 722.

To achieve left atrial and ventricular sensing, pacing and shocking, the connector includes at least a left ventricular tip terminal (V_{L} TIP) 844, a left atrial ring terminal (A_{L} RING) 846, and a left atrial shocking terminal (A_{L} COIL) 848, which are adapted for connection to the left ventricular ring electrode 726, the left atrial tip electrode 727, and the left atrial coil electrode 728, respectively.

To support right ventricle sensing, pacing and shocking, the connector further includes a right ventricular tip terminal (V_{R} TIP) 852, a right ventricular ring terminal (V_{R} RING) 854, a right ventricular shocking terminal (R_{V} COIL) 856, and an SVC shocking terminal (SVC COIL) 858, which are adapted for connection to the right ventricular tip electrode 732, right ventricular ring electrode 734, the RV coil electrode 726, and the SVC coil electrode 738, respectively.

An atrial pulse generator 870 and a ventricular pulse generator 872 generate pacing stimulation pulses for delivery by the right atrial lead 720, the right ventricular lead 730, and/or the coronary sinus lead 724 via an electrode configuration switch 874. It is understood that in order to provide stimulation therapy in each of the four chambers of the heart, the atrial and ventricular pulse generators, 870 and 872, may include dedicated, independent pulse generators, multiplexed pulse generators, or shared pulse generators. The pulse generators, 870 and 872, are controlled by the microcontroller 860 via appropriate control signals, 876 and 878, respectively, to trigger or inhibit the stimulation pulses.

The microcontroller 860 further includes timing control circuitry 879 which is used to control pacing parameters (e.g., the timing of stimulation pulses) as well as to keep track of the timing of refractory periods, noise detection windows, evoked response windows, alert intervals, marker channel timing, etc., which is well known in the art. Examples of pacing parameters include, but are not limited to, atrio-ventricular delay, interventricular delay and interatrial delay.

The switch bank 874 includes a plurality of switches for connecting the desired electrodes to the appropriate I/O circuits, thereby providing complete electrode programmability. Accordingly, the switch 874, in response to a control signal 880 from the microcontroller 860, determines the polarity of the stimulation pulses (e.g., unipolar, bipolar, etc.) by selectively closing the appropriate combination of switches (not shown) as is known in the art.

Atrial sensing circuits 882 and ventricular sensing circuits 884 may also be selectively coupled to the right atrial lead 720, coronary sinus lead 724, and the right ventricular lead 730, through the switch 874 for detecting the presence of cardiac activity in each of the four chambers of the heart. Accordingly, the atrial (ATR. SENSE) and ventricular (VTR. SENSE) sensing circuits, 882 and 884, may include dedicated sense amplifiers, multiplexed amplifiers, or shared amplifiers. The switch 874 determines the “sensing polarity” of the cardiac signal by selectively closing the appropriate switches, as is also known in the art. In this way, the clinician may program the sensing polarity independent of the stimulation polarity.

Each sensing circuit, 882 and 884, preferably employs one or more low power, precision amplifiers with programmable gain and/or automatic gain control, bandpass filtering, and a threshold detection circuit, as known in the art, to selectively sense the cardiac signal of interest. The automatic gain control enables the device 710 to deal effectively with the difficult problem of sensing the low amplitude signal characteristics of atrial or ventricular fibrillation. Such sensing circuits, 882 and 884, can be used to determine cardiac performance values used in the present invention. Alternatively, an automatic sensitivity control circuit may be used to effectively deal with signals of varying amplitude.

The outputs of the atrial and ventricular sensing circuits, 882 and 884, are connected to the microcontroller 860 which, in turn, are able to trigger or inhibit the atrial and ventricular pulse generators, 870 and 872, respectively, in a demand fashion in response to the absence or presence of cardiac activity, in the appropriate chambers of the heart. The sensing circuits, 882 and 884, in turn, receive control signals over signal lines, 886 and 888, from the microcontroller 860 for purposes of measuring cardiac performance at appropriate times, and for controlling the gain, threshold, polarization charge removal circuitry (not shown), and timing of any blocking circuitry (not shown) coupled to the inputs of the sensing circuits, 882 and 886.

For arrhythmia detection, the device 710 includes an arrhythmia detector 862 that utilizes the atrial and ventricular sensing circuits, 882 and 884, to sense cardiac signals to determine whether a rhythm is physiologic or pathologic. The timing intervals between sensed events (e.g., P-waves, R-waves, and depolarization signals associated with fibrillation) can be classified by the microcontroller 860 by comparing them to a predefined rate zone limit (i.e., bradycardia, normal, low rate VT, high rate VT, and fibrillation rate zones) and various other characteristics (e.g., sudden onset, stability, physiologic sensors, and morphology, etc.) in order to assist with determining the type of remedial therapy that is needed (e.g., bradycardia pacing, anti-tachycardia pacing, cardioversion shocks or defibrillation shocks, collectively referred to as “tiered therapy”). Additionally, the arrhythmia detector 862 can perform arrhythmia discrimination, e.g., using measures of arterial blood pressure determined in accordance with embodiments of the present invention. Exemplary details of such arrhythmia discrimination, including tachyarrhythmia classification, are discussed above. The arrhythmia detector 862 can be implemented within the microcontroller 860, as shown in FIG. 8. Thus, this detector 862 can be implemented by software, firmware, or combinations thereof. It is also possible that all, or portions, of the arrhythmia detector 862 can be implemented using hardware. Further, it is also possible that all, or portions, of the arrhythmia detector 862 can be implemented separate from the microcontroller 860.

In accordance with embodiments of the present invention, the implantable device 710 includes an arterial stiffness monitor 867, which can monitor arterial stiffness using the techniques described above with reference to FIGS. 1-6. The arterial stiffness monitor 867 can be implemented within the microcontroller 860, as shown in FIG. 8, and can be implemented by software, firmware, or combinations thereof. It is also possible that all, or portions, of the arterial stiffness monitor 867 to be implemented using hardware. Further, it is also possible that all, or portions, of the arterial stiffness monitor 867 to be implemented separate from the microcontroller 860.

The implantable devices 710 can be used to provide therapy based on the arterial stiffness determined by the arterial stiffness monitor 867. For example, the pacing can be provided through one or more of leads 842, 844, 846, 848, 852, 856 and 858 can be adjusted. In a further embodiment, based on detected arterial stiffness or trends in a measure associated with arterial stiffness, medication, such as statins, can be provided through medication pump 803 or externally.

The arterial stiffness monitor 867 can be used in a closed loop control system to detect changes in the arterial stiffness in response to changed pacing parameters or some other therapy so as to adjust the arterial stiffness.

The implantable device 710 is also shown as including an activity and/or posture sensor 827. Such a sensor 827 can be a simple one dimensional sensor that converts mechanical motion into a detectable electrical signal, such as a back electro magnetic field (BEMF) current or voltage, without requiring any external excitation. Alternatively, the sensor 827 can measure multi-dimensional activity information, such as two or more of acceleration, direction, posture and/or tilt. Additional details of exemplary multi-dimensional activity sensors, as well as how they can be used when monitoring arterial stiffness in accordance with embodiments of the present invention, were discussed above with reference to the flow diagrams of FIGS. 1-3.

The implantable device 710 can include a pacing controller 866, which can adjust a pacing rate and/or pacing intervals based on measures of arterial blood pressure, in accordance with embodiments of the present invention. The pacing controller 866 can be implemented within the microcontroller 860, as shown in FIG. 8. Thus, the pacing controller 866 can be implemented by software, firmware, or combinations thereof. It is also possible that all, or portions, of the pacing controller 866 can be implemented using hardware. Further, it is also possible that all, or portions, of the pacing controller 866 can be implemented separate from the microcontroller 860.

Still referring to FIG. 8, cardiac signals are also applied to the inputs of an analog-to-digital (ND) data acquisition system 890. The data acquisition system 890 is configured to acquire IEGM and/or ECG signals, convert the raw analog data into a digital signal, and store the digital signals for later processing and/or telemetric transmission to an external device 802. The data acquisition system 890 can be coupled to the right atrial lead 720, the coronary sinus lead 724, and the right ventricular lead 730 through the switch 874 to sample cardiac signals across any pair of desired electrodes.

The data acquisition system 890 can be coupled to the microcontroller 860, or other detection circuitry, for detecting an evoked response from the heart 712 in response to an applied stimulus, thereby aiding in the detection of “capture”. Capture occurs when an electrical stimulus applied to the heart is of sufficient energy to depolarize the cardiac tissue, thereby causing the heart muscle to contract. The microcontroller 860 detects a depolarization signal during a window following a stimulation pulse, the presence of which indicates that capture has occurred. The microcontroller 860 enables capture detection by triggering the ventricular pulse generator 872 to generate a stimulation pulse, starting a capture detection window using the timing control circuitry 879 within the microcontroller 860, and enabling the data acquisition system 890 via control signal 892 to sample the cardiac signal that falls in the capture detection window and, based on the amplitude, determines if capture has occurred.

The implementation of capture detection circuitry and algorithms are well known. See for example, U.S. Pat. No. 4,729,376 to Decote, Jr.; entitled “Cardiac Pacer and Method Providing Means for Periodically Determining Capture Threshold and Adjusting Pulse Output Level Accordingly”, U.S. Pat. No. 4,708,142, to Decote, Jr., entitled “Automatic Cardiac Capture Threshold Determination System and Method”; U.S. Pat. No. 4,686,988, to Sholder, entitled “Pacemaker System and Method for Measuring and Monitoring Cardiac Activity and for Determining and Maintaining Capture”; U.S. Pat. No. 4,969,467 to Callaghan et al.; entitled “Pacemaker with Improved Automatic Output Regulation” and U.S. Pat. No. 5,350,410 to Kleks et al., entitled “Autocapture System for Implantable Pulse Generator”, which patents are hereby incorporated herein by reference. The type of capture detection system used is not critical to the present invention.

The microcontroller 860 is further coupled to the memory 894 by a suitable data/address bus 896, wherein the programmable operating parameters used by the microcontroller 860 are stored and modified, as required, in order to customize the operation of the implantable device 710 to suit the needs of a particular patient. Such operating parameters define, for example, pacing pulse amplitude, pulse duration, electrode polarity, rate, sensitivity, automatic features, arrhythmia detection criteria, and the amplitude, wave shape and vector of each shocking pulse to be delivered to the patient's heart 712 within each respective tier of therapy. The memory 894 can also store data about arterial stiffness, as determined using embodiments of the present invention.

The operating parameters of the implantable device 710 may be non-invasively programmed into the memory 894 through a telemetry circuit 801 in telemetric communication with an external device 802, such as a programmer, transtelephonic transceiver, or a diagnostic system analyzer. The telemetry circuit 801 can be activated by the microcontroller 860 by a control signal 806. The telemetry circuit 801 advantageously allows intracardiac electrograms and status information relating to the operation of the device 710 (as contained in the microcontroller 860 or memory 894) to be sent to the external device 802 through an established communication link 805. The telemetry circuit can also be use to transmit data relating to arterial stiffness, such as alarms and measurement data, to the external device 802.

For examples of telemetry devices, see U.S. Pat. No. 4,809,697 entitled “Interactive Programming and Diagnostic System for use with Implantable Pacemaker” to Causey, III et al.; U.S. Pat. No. 4,944,299 to Silvian, entitled “High Speed Digital Telemetry System for Implantable Device”; and U.S. Pat. No. 6,275,734 to McClure et al., entitled “Efficient Generation of Sensing Signals in an Implantable Medical Device such as a Pacemaker or ICD”, which are hereby incorporated herein by reference.

The implantable device 710 additionally includes a battery 811 which provides operating power to all of the circuits shown in FIG. 8. If the implantable device 710 also employs shocking therapy, the battery 811 should be capable of operating at low current drains for long periods of time, and then be capable of providing high-current pulses (for capacitor charging) when the patient requires a shock pulse. The battery 811 should also have a predictable discharge characteristic so that elective replacement time can be detected.

The implantable device 710 can also include a magnet detection circuitry (not shown), coupled to the microcontroller 860. It is the purpose of the magnet detection circuitry to detect when a magnet is placed over the implantable device 710, which magnet may be used by a clinician to perform various test functions of the implantable device 710 and/or to signal the microcontroller 860 that the external programmer 802 is in place to receive or transmit data to the microcontroller 860 through the telemetry circuits 801.

As further shown in FIG. 8, the device 710 is also shown as having an impedance measuring circuit 813 which is enabled by the microcontroller 860 via a control signal 814. The known uses for an impedance measuring circuit 813 include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds and heart failure condition; detecting when the device has been implanted; measuring stroke volume; and detecting the opening of heart valves, etc. The impedance measuring circuit 813 is advantageously coupled to the switch 874 so that any desired electrode may be used. The impedance measuring circuit 813 can be used to obtain an impedance plethysmography (IPG) signal, which can be used in certain embodiments of the present invention to monitor arterial stiffness.

In the case where the implantable device 710 is also intended to operate as an implantable cardioverter/defibrillator (ICD) device, it should detect the occurrence of an arrhythmia, and automatically apply an appropriate electrical shock therapy to the heart aimed at terminating the detected arrhythmia. To this end, the microcontroller 860 further controls a shocking circuit 816 by way of a control signal 818. The shocking circuit 816 generates shocking pulses of low (up to 0.5 Joules), moderate (0.5-10 Joules), or high energy (11 to 40 Joules), as controlled by the microcontroller 860. Such shocking pulses are applied to the patient's heart 712 through at least two shocking electrodes, and as shown in this embodiment, selected from the left atrial coil electrode 728, the RV coil electrode 736, and/or the SVC coil electrode 738. As noted above, the housing 740 may act as an active electrode in combination with the RV electrode 736, or as part of a split electrical vector using the SVC coil electrode 738 or the left atrial coil electrode 728 (i.e., using the RV electrode as a common electrode).

The above described implantable device 710 was described as an exemplary pacing device. One or ordinary skill in the art would understand that embodiments of the present invention can be used with alternative types of implantable devices. Accordingly, embodiments of the present invention should not be limited to use only with the above described device.

The implantable sensor can also be part of a stand alone device that is not part of a pacing system. Such a stand alone device can monitor arterial stiffness and output indications of the arterial stiffness.

The present invention has been described above with the aid of functional building blocks illustrating the performance of specified functions and relationships thereof. The boundaries of these functional building blocks have often been arbitrarily defined herein for the convenience of the description. Alternate boundaries can be defined so long as the specified functions and relationships thereof are appropriately performed. Any such alternate boundaries are thus within the scope and spirit of the claimed invention. For example, it would be possible to combine or separate some of the steps shown in the flow diagrams. Further, it may be possible to change the order of some of the steps shown in flow diagrams without substantially changing the overall events and results. For another example, it is possible to change the boundaries of some of the blocks shown in FIG. 8.

The previous description of the preferred embodiments is provided to enable any person skilled in the art to make or use the embodiments of the present invention. While the invention has been particularly shown and described with reference to preferred embodiments thereof, it will be understood by those skilled in the art that various changes in form and details may be made therein without departing from the spirit and scope of the invention.

What is claimed is:

1. For use with an implanted system, a method for monitoring a patient's arterial stiffness, comprising:
(a) using an implanted sensor to produce a signal indicative of changes in arterial blood volume for a plurality of beats of the patient's heart;
(b) determining a pulse duration metric for each of a plurality of pulses of the signal, wherein each pulse of the signal corresponds to a beat of the patient's heart; and
(c) monitoring arterial stiffness based on the determined pulse duration metric for the plurality of pulses of the signal.

2. The method of claim 1, wherein step (c) comprises:
(c.i) determining, based on the results of step (b), a measure of dispersion of the pulse duration metric; and
(c.ii) monitoring arterial stiffness based on the measure of the dispersion.

3. The method of claim 2, wherein the measure of dispersion of the pulse duration metric is selected from the group consisting of:
standard deviation,
normalized standard deviation,
interquartile range,
range,
mean difference,
median absolute deviation,
average absolute deviation
coefficient of variation,
quartile coefficient of dispersion,
relative mean difference,
variance, and
variance-to-mean ratio of the metric.

4. The method of claim 2, wherein the measure of dispersion of the pulse duration metric is the normalized standard deviation of the pulse duration metric.

5. The method of claim 2, wherein step (c.ii) comprises:
comparing the measure of dispersion of the pulse duration metric to a threshold;
detecting an abnormal arterial stiffness when the measure of dispersion falls below the threshold; and
triggering an alarm and/or therapy and/or adjusting therapy in response to detecting an abnormal arterial stiffness.

6. The method of claim 2, wherein:
steps (a), (b) and (c.i) are performed for each of a plurality of periods of time to thereby determine a measure of dispersion of the pulse duration metric for each of the plurality of periods of time; and
step (c.ii) comprises monitoring changes in arterial stiffness based on changes in the measure of dispersion;
wherein a decrease in the measure of dispersion is indicative of an increase in arterial stiffness.

7. The method of claim 6, further comprising:
(d) determining and storing information indicative of arterial stiffness burden.

8. The method of claim 1, wherein step (b) comprises determining the pulse duration metric for the plurality of pulses of the signal by determining, for each of the plurality of pulses of the signal, a duration of the pulse of the signal at a percentage of a peak-to-peak amplitude of the pulse for the beat.

9. The method of claim 10, wherein the percentage of a peak-to-peak amplitude of the pulse, at which the duration is determined, is in the range of 30% to 90%.

10. The method of claim 1, wherein step (c) comprises:
(c.i) determining, based on the results of step (b), an average of the pulse duration metric; and
(c.ii) monitoring arterial stiffness based on the average of the pulse duration metric.

11. The method of claim 10, wherein:
steps (a), (b) and (c.i) are performed for each of a plurality of periods of time to thereby determine an average of the pulse duration metric for each of the plurality of periods of time; and
step (c.ii) comprises monitoring changes in arterial stiffness based on changes in the average of the pulse duration metric;
wherein a decrease in the average of the pulse duration metric is indicative of an increase in arterial stiffness.

12. The method of claim 1, wherein step (a) comprises using an implanted photoplethysmography (PPG) sensor to obtain a photoplethysmography (PPG) signal indicative of changes in arterial blood volume, or using an implanted impedance plethysmography (IPG) sensor to obtain an impedance plethysmography (IPG) signal indicative of changes in arterial blood volume.

13. An implantable system, comprising:
an implantable sensor configured to produce a signal that is indicative of changes in arterial blood volume for a plurality of beats of a patient's heart; and
a monitor configured to:
determine a pulse duration metric for each of a plurality of pulses of the signal, wherein each pulse of the signal corresponds to a beat of the patient's heart; and
monitor arterial stiffness based on the determined pulse duration metric for the plurality of pulses of the signal.

14. The implantable system of claim 13, wherein the monitor is configured to determine a measure of dispersion of the pulse duration metric and to monitor arterial stiffness based on the measure of the dispersion.

15. The implantable system of claim 14, wherein the measure of dispersion of the pulse duration metric is selected from the group consisting of:
standard deviation,
normalized standard deviation,
interquartile range,
range,
mean difference,
median absolute deviation,
average absolute deviation
coefficient of variation,
quartile coefficient of dispersion,
relative mean difference,
variance, and
variance-to-mean ratio of the metric.

16. The implantable system of claim 14, wherein the measure of dispersion of the pulse duration metric is the normalized standard deviation of the pulse duration metric.

17. The implantable system of claim 14, wherein the monitor is configured to compare the measure of dispersion of the pulse duration metric to a threshold, detect an abnormal arterial stiffness when the measure of dispersion falls below the threshold, and trigger an alarm and/or therapy and/or adjust therapy in response to detecting an abnormal arterial stiffness.

18. The implantable system of claim 13, wherein the monitor is configured to determine the pulse duration metric for the plurality of pulses of the signal by determining, for each of the plurality of pulses of the signal, a duration of a pulse of the signal at a percentage of a peak-to-peak amplitude of the pulse for the beat.

19. The implantable system of claim 13, wherein the monitor is configured to determine an average of the pulse duration metric for the plurality of pulses of the signal, and monitor arterial stiffness based on the average.

20. The implantable system of claim 13, wherein the implantable sensor comprises an implanted photoplethysmography (PPG) sensor configured to obtain a photoplethysmography (PPG) signal indicative of changes in arterial blood volume, or an implanted impedance plethysmography (IPG) sensor configured to obtain an impedance plethysmography (IPG) signal indicative of changes in arterial blood volume.
